(19) United States
(12) Patent Application Publication
(10) Pub. No.: US 2004/0204482 A1
(43) Pub. Date: Oct. 14, 2004

(54) Extended release oral dosage form

(76) Inventors: Karin Ellstrom, Sodertalje (SE); Ulf Kjellberg, Sodertalje (SE); Hakan Nyqvist, Sodertalje (SE); Annika Schweighofer, Sodertalje (SE); Maritta Temsamani, Sodertalje (SE)

Correspondence Address: WHITE & CASE LLP, PATENT DEPARTMENT, 1155 AVENUE OF THE AMERICAS, NEW YORK, NY, 10036, US

(21) Appl. No.: 10/488,022

(22) PCT Filed: Aug. 28, 2002
(86) PCT No.: PCT/SE02/01540

(30) Foreign Application Priority Data
Aug. 29, 2001 (SE) 0102886-9

(51) Int. Cl. A61K 31/343; A61K 9/22

(52) U.S. Cl. 514/469; 424/468

(57) ABSTRACT

The present invention relates to an extended release oral dosage form of a pharmaceutically active substance, (R)-3-N,N-dicyclobutylamino-8-fluoro-3,4-dihydro-2H-1-benzopyran-5-carboxamide, in the form of the free base or pharmaceutically acceptable salts and/or hydrates or solvates thereof. Furthermore, the invention relates to an extended release oral dosage form that provides a defined blood concentration profile having no rapid initial rise in blood plasma concentration of said active substance when administered at low dose. The invention further relates to processes for preparing said dosage form, the use of said dosage form and a method of prevention and/or treatment of CNS disorders and related medical disturbances using said dosage form.

### FIELD OF THE INVENTION

The present invention relates to an extended release oral dosage form of a pharmaceutically active substance, (R)-3-N,N-dicyclobutylamino-8-fluoro-3,4-dihydro-2H-1-benzopyran-5-carboxamide, in the form of the free base or pharmaceutically acceptable salts and/or hydrates or solvates thereof, for use in the prevention and/or treatment of central nervous system (CNS) disorders and related medical disturbances, especially 5-hydroxytryptamine mediated disorders and disturbances. More particularly, the invention relates to an extended release oral dosage form that provides a defined blood concentration profile having no rapid initial rise in blood plasma concentration of said active substance when administered at low dose. The invention further relates to processes for preparing said dosage form, the use of said dosage form for the manufacture of a medicament and a method of prevention and/or treatment of CNS disorders and related medical disturbances using said dosage form.

### BACKGROUND OF THE INVENTION

The development of new pharmaceutical active substances is often hampered or even blocked due to side effects of these new active substances. Some of the side effects may be overcome by developing suitable pharmaceutical formulations. This is for example true for substances that have a blood plasma concentration profile that starts with a rapid initial rise in blood plasma concentration which creates an early sharp and high peak plasma concentration. This early and high peak plasma concentration of the active substance can cause severe side effects. This problem can be overcome by altering the blood plasma concentration profile so that a more gradual absorbtion rate is obtained.

Also, a short half-life of active substances can lead to low and insufficient concentrations in the blood plasma of the active substances at the end of a dosage interval. Increasing the dose can overcome this low and insufficient blood concentration. However, it is well known that administration of large doses of an active substance increases the risk for side effects. This is especially true for active substances that have their site of action in the brain. Administration of large doses of an anti-depressant drug can, for example, cause side effects such as dizziness, nausea, vomiting, etceteras. In the prevention and/or treatment of CNS disorders and related medical disturbances, the use of good soluble active substances having a short half-life and/or having a rapid initial rise in blood plasma concentration may therefore be limited.

Extended release dosage forms can be used to overcome these problems.

An extended release oral dosage form makes it possible to deliver an active. substance to the blood in a controlled way such that the initial sharp rise in blood plasma concentration of the active substance is avoided. Another advantage of extended release oral dosage forms is the possibility to administer the prescribed daily dose of the active substance in the form of one unit dose while maintaining the desired therapeutic response over a period of up to 24 hours. In this way, the administration will be more user-friendly. Furthermore, the risk for therapeutic inefficiency due to bad compliance to frequent dosing and the lack of dosing during the night can be minimised. Extended release oral dosage forms further have the potential for improving treatment of e.g. chronic diseases. Besides, both systemic and local side effects, e.g. gastrointestinal irritation due to high local concentrations of the active substance, can be reduced.

A lot of research has been done with regard to the development of new extended release oral dosage forms. This has resulted in complicated formulations such as for instance entirely or partly coated multi-layer tablets containing the active substance in different amounts in the different layers and/or coating. In spite of all these developments, in general, extended release oral dosage forms still have some disadvantages. Development of an extended release oral dosage form whereby the time of release for a good soluble active substance can be varied is still a challenge. Furthermore, good soluble active substances with a short half-life are difficult to formulate in an extended release oral dosage form using a gel-forming polymer. Another problem is that the rate and/or extent of bioavailabilty are often influenced by the different physiological conditions of the gastrointestinal tract such as pH, enzyme activity and food intake. Especially food intake may cause problems with regard to the rate and/or extent of bioavailabilty. Another problem is related to dose dumping, i.e. a large amount of the dose is released in a short period of time.

### BRIEF DESCRIPTION OF THE INVENTION

It has now surprisingly been found that the pharmaceutical extended release oral dosage form of the present invention avoids the above-mentioned problems. The present invention provides for an extended release dosage form which is especially suitable for a good soluble active substance comprising a homogeneous mixture of the good soluble active substance, a gel-forming polymer and optionally other excipients, whereby the amount of the active substance is preferably low in the dosage form (<10% w/w). The dosage form may be coated or uncoated. The oral dosage form of the present invention provides for a defined release profile of the active substance in vivo over a predetermined period of up to 24 hours, whereby also the rapid initial rise in blood plasma concentration of the active substance is avoided. This means that there are less fluctuations of the blood plasma concentration over time and thus less risk for adverse effects due to such fluctuations as compared to an immediate release pharmaceutical dosage form. The prolonged and controlled plasma level of the active substance makes it possible to administer the active substance once or twice a day, thereby increasing patient compliance.

The active substances for use in the present invention are pharmaceutically good soluble active substances, which may for example be used in the prevention and/or treatment of disorders and related disturbances in the CNS. The present invention is especially suitable for substances having a short half-life and/or a rapid initial rise in blood plasma concentration. Of particular interest is the 5-hydroxytryptamine receptor antagonist (R)-3-N,N-dicyclobutylamino-8-fluoro-3,4-dihydro-2H-1-benzopyran-5-carboxamide in the form of the free base or pharmaceutically acceptable salts and/or hydrates or solvates thereof. Particularly, the salt (R)-3-N,N-dicyclobutylamino-8-fluoro-3,4-dihydro-2H-1-benzopyran-5-carboxamide hydrogen tartrate. This tartrate comprises any of the optical forms (2R,3S), (2R,3R) and (2S,3S). Of these forms (R)-3-N,N-dicyclobutylamino-8-fluoro-3,4-dihydro-2H-1-benzopyran-5-carboxamide hydrogen (2R,3R)-tartrate is preferred. The most preferred substance is (R)-3-N,N-dicyclobutylamino-8-fluoro-3,4-dihydro-2H-1-benzopyran-5-carboxamide hydrogen (2R,3R)-tartrate monohydrate (J. of Pharm. and Exp. Ther. (1997) Vol 283 No. 1, pp 216-225), which has a water solubility of 70 mg/ml at 25° C. and a plasma elimination half-life, t_{1/2} of 1.5 hours.

The time to reach the maximum blood plasma concentration (tₘₐₓ), after oral administration of the active substance in the extended release oral dosage form of the present invention, should be at least five times as long as the tₘₐₓ of the same substance when administered orally in an aqueous solution, under fasting conditions. The tₘₐₓ may be between 3 to 7 hours, and is preferable at least 3 hours.

Other parameters that can be used to express the blood plasma concentration profile of the active substance provided for by the extended release oral dosage form of the present invention are the area under the curve (AUC), the mean residence time (MRT) and the relative bioavailability (Fᵣₑₗ). The MRT of an active substance reflects the mean time a molecule resides in the body. The MRT of the active substance in the extended dosage form of the present invention should be at least three times as long as the MRT of the same substance when administered orally in an aqueous solution. The MRT is preferable between 8and 15 hours, most preferably between 8 and 13 hours. An aqueous solution shall mean a water solution containing the active substance.

Further, the present invention relates to an extended release oral dosage form, which provides therapeutic levels of the active substance in blood plasma for at least 24 hours, and which has an in vitro dissolution profile in a phosphate buffer, pH 6.8, using USP Paddle method at 50 rpm, such that about 30 to 75% of the active substance is released after 4 hours, about 60 to 100% is released after 12 hours and about 80 to 100% is released after 24 hours.

Particularly, the present invention relates to an extended release oral dosage form, which provides therapeutic levels of the active substance in blood plasma for at least 24 hours, and which has an in vitro dissolution profile in a phosphate buffer, pH 6.8, using USP Paddle method at 50 rpm, such that about 30 to 55% of the active substance is released after 4 hours, about 60 to 90% is released after 12 hours and about 80 to 100% is released after 24 hours.

The present invention further provides for an extended release oral dosage form, whereby the time of release of the active substance easily can be adjusted by using a release modifying agent, e.g. lactose. The present invention also relates to an extended release oral dosage form, which provides therapeutic levels of the active substance in blood plasma for at least 12 hours and which has an in vitro dissolution profile in a phosphate buffer, pH 6.8, using USP Paddle method at 50 rpm, such that about 56 to 75% of the active substance is released after 4 hours, about 70 to 95% is released after 8 hours and about 90 to 100% is released after 12 hours.

The dosage form of the invention shall contain at least one gel-forming polymer, which may be selected from the group of hydroxymethyl cellulose, hydroxypropyl cellulose, hydroxyethyl cellulose, hydroxypropyl methylcellulose, methyl cellulose, ethyl cellulose, polyvinylpyrrolidone, polyethylene glycols, polyethylene oxide and poloxamers.

Preferably, the gel-forming polymer is hydroxypropyl methylcellulose (HPMC). The viscosity of the HPMC should be between 3000 and 21000 cP, preferably 7500 and 21000 cP, and most preferably 11250 and 21000 cP of a 2% (w/w) aqueous solution at 20° C. The substitution degree of methoxy groups of this cellulose should be from 19 to 30% by weight, preferably from 19 to 28% and most preferably from 19 to 24% by weight and the substitution degree of hydroxypropoxy groups should be from 4 to 12% by weight, most preferably 7 to 12% by weight

This HPMC may be mixed with a low viscosity HPMC. The low viscosity cellulose should have a viscosity within 3.75 and 140 cP, preferably from 11.3 to 140 cP and most preferably from 37.5 to 70 cP of a 2% (w/w) aqueous solution at 20° C. The substitution degree of methoxy groups of this cellulose should be from 19 to 30% by weight, preferably from 19 to 28% and most preferably from 19 to 24% by weight. The substitution degree of hydroxypropoxy groups should be from 4 to 12% by weight, most preferably 7 to 12% by weight.

The ratio of active substance to gel-forming polymer in the extended release oral dosage form of the present invention may be from 1:10 to 1:60, preferably from 1:30to 1:60.

Beside the gel-forming polymers, the dosage form may optionally comprise excipients, such as binders, release modifying agents, lubricants, flow condition agents and the like. Suitable binders are hydroxypropyl cellulose, microcrystalline cellulose, polyvinylpyrrolidone, gelatine, polyethylene glycol, glycerylbehenate, glycerylmonostearate, carnauba wax and the like. The preferred binders are hydroxypropyl cellulose, polyvinylpyrrolidone (PVP) and microcrystalline cellulose. The amount of binder in the composition is from 0 to 25% w/w.

The ratio of active substance to binder may be from 1:0.5 to 1:5, preferably from 1:0.7.

Other excipients that may be used in the formulation are lubricants, such as magnesium stearate, sodium stearyl fumarate, stearic acid, polyethylene glycol, talc and the like, flow condition agents, such as colloidal silicon dioxide, talc and the like. As further excipients for the modification of the release, either slower or faster, can be mentioned lactose, mannitol, sorbitol, calcium phosphate, aluminium silicate, paraffin, carboxypolymethylene, carboxyvinyl polymer, acrylic acid polymer, ethyl cellulose, polyethylene glycol and the like. Additionally, excipients such as taste agents and colouring agents may be used. The amounts of these excipients are 0 to 55% w/w.

The dosage form may be prepared by mixing the active substance, the gel-forming polymer and optionally other excipients such as binders, lubricants and the like in a suitable mixer, e.g. a Turbula mixer, followed by direct compression of said homogeneous mixture. Alternatively, the dosage form may be prepared from a granulated powder. The homogeneous powder mixture may be obtained by mixing the active substance, the gel-forming polymer and optionally excipients such as binders in a suitable mixer. Then the mixture may be granulated in water or another granulation liquid such as an alcohol, e.g. ethanol, methanol, isopropanol, a ketone, e.g. acetone or aqueous mixtures thereof. From an environmental point of view water is preferred. The resultant wet granulation may thereafter be dried in a drying cabinet or in a fluid bed dryer and milled through a screen. The granulation may also be performed at elevated temperatures by using meltable binders. The cooled granulation may be milled through a screen. The dry granulate powder mass is then mixed with other excipients and compressed into a suitable dosage form.

Thirdly, the dosage form may be prepared by first compressing the dry granulate powder mass into loose compacts. These loose compacts may be milled through a screen and finally mixed with other excipients such as binders, lubricants and flow condition agents. The dry homogeneous powder mass may then be manufactured into a suitable dosage form, e.g. compressed into tablets in a tablet machine. Other suitable oral dosage forms are capsules, minitablets and the like.

The dosage form of the present invention may further comprise a coating layer. Suitable polymers that can be used in the coating layer are hydroxypropyl cellulose, low viscosity hydroxypropyl methylcellulose, polyethylene glycol. The coating layer may further s comprise binders such as microcrystalline cellulose, hydroxypropyl cellulose and the like, plastizicers such as polyethylene glycol, acetyl tributyl citrate and the like and colour agents such as titanium dioxide, iron oxide and the like. Also, antiadhesion agents such as colloid silicon dioxide, talc and the like may be used in the coating layer. The coating layer may additionally comprise taste-masking agents. As coating fluid may be used water or alcohols such as ethanol, optionally containing antibacterial agents such as hydrogen peroxide. The coating layer may be applied by way of spray coating in a fluidised bed, pan-coating or another coating technique known to a person skilled in the art.

In one embodiment of the present invention the extended release oral dosage form is uncoated.

The composition from which the dosage form is prepared can be formulated to contain the active substance in different amounts, e.g. between 0.1 and 50 mg, preferably between 1 and 25 mg, but is not limited to these intervals. Suitable daily doses of the active substance may vary within a wide range and will depend on various factors such as the relevant disorder or medical conditions, the age, weight and sex, and may be determined by a physician.

The dosage form according to the invention can be used in the prevention and/or treatment of CNS disorders and related medical disturbances, urinary incontinence, vasospasm or growth control of tumours, particularly for 5-hydroxytryptamine mediated disorders and medical disturbances. Further, the extended release dosage form could for example be used in the prevention and/or treatment of affective disorders, mood disorders e.g. depression, major depressive episodes, dysthymia, seasonal affective disorder, depressive phases of bipolar disorder, anxiety disorders e.g. obsessive compulsive disorder, panic disorder with/without agoraphobia, social phobia, specific phobia, generalised anxiety disorder, posttraumatic stress disorder, personality disorders e.g. disorders of impulse control, trichotellomania, sleep disorders, eating disorders e.g. obesity, anorexia, bulimia, pre-menstrual syndrome, sexual disturbances, alcoholism, tobacco abuse, autism, attention deficit, hyperactivity disorder, migraine, memory disorders e.g. age associated memory impairment, presenile and senile dementia such as Alzheimer's disease, pathological aggression, schizophrenia, endocrine disorders e g hyperprolactinaemia, stroke, dyskinesia, Parkinson's disease, thermoregulation, pain, hypertension, urinary incontinence such as over active bladder, detrusor instability, neurogenic bladder, detrusor hyperreflexia, nocturnal enuresis, e.g. bed-wetting in children, urinary frequency, urinary urgency, urge incontinence, stress incontinence, mixed incontinence, unstable bladder secondary to prostatitis or interstitial cystitis, disturbances of the cardiovascular system and disturbances in the gastrointestinal system.

The present invention also relates to processes for the manufacture of the extended release dosage form characterized by,

Method A, comprising the steps:

Ai) mixing the active substance with the gel-forming polymers and optionally binders, lubricants, modifying agents and other excipients,

Aii) forming the obtained dry powder mixture into a suitable solid dosage form and

Aiii) optionally coating the obtained dosage form,

or,

Method B, comprising the steps:

Bi) mixing the active substance with the gel-forming polymers and optionally binders and other excipients,

Bii) granulating said mixture,

Biii) optionally drying the obtained granulate,

Biv) mixing the granulate with other excipients,

Bv) forming the obtained dry powder mixture into a suitable solid dosage form and

Bvi) optionally coating the obtained dosage form,

or,

Method C, comprising the steps:

Ci) mixing the active substance with the gel-forming polymers and optionally binders and other excipients,

Cii) granulating said mixture,

Ciii) optionally drying the obtained granulate,

Civ) compressing the granulate powder mass into loose compacts,

Cv) milling the compacts and mixing them with other excipients,

Cvi) forming the obtained dry powder mixture into a suitable solid dosage form and

Cvii) optionally coating the obtained dosage form.

The term ‘extended release oral dosage form’ shall mean any oral dosage form, which continuously releases the active substance at rates, which are sufficient to provide periods of prolonged therapeutic action following each administration of a single dose of such a dosage form. Alternative naming is e.g. controlled, sustained and slow release.

According to the Biopharmaceutical Classification System used by the FDA, the term ‘good soluble’ shall means, the maximum dose to be administered, should be able to dissolve in 250 ml of an aqueous solution in the pH range of 1 to 8. The aqueous solution is preferably water.

Abbreviations;

| | |
|---|---|
| CNS | Central Nervous System |
| Cₘₐₓ | maximum blood plasma concentration (nmol/L) |
| C₂₄ | blood plasma concentration after 24 hours (nmol/L) |
| Cₜ | blood plasma concentration after t hours (nmol/L) |
| t | time (h) |
| t_{1/2} | plasma elimination half-life (h) |
| tₘₐₓ | time to reach the maximum blood plasma concentration (h) |
| AUC | Area Under the plasma concentration |
| | versus time Curve (nmol * h/L) |
| MRT | Mean Residence Time (h) |
| Fᵣₑₗ | relative bioavailability |
| n | number of persons participating in clinical tests |
| ER | Extended Release |
| HPMC | Hydroxypropyl methylcellulose |
| HPC (LF) | Hydroxypropylcellulose (molecular weight .approx. 95,000, |
| | pharmaceutical grade) |
| PEG | Polyethylene glycol |
| ATBC | Acetyl tributylcitrate |
| PVP | Polyvinylpyrrolidone |
| HPLC | High Pressure Liquid Chromatography |

### EXAMPLES

The invention will now be illustrated by the following non-limiting examples.

### Example 1

The following components, expressed as mg per tablet, were used; batch size

| | |
|---|---|
| Active substance: | 5.00 |
| HPC LF | 3.5 |
| HPMC (15000 cP) | 138.00 |
| HPMC (50 cP) | 59.2 |
| Water | 110.20 |
| Magnesium stearate | 3.2 |
| Colloidal silicon dioxide | 1.1 |

HPMC (15000 cP) has a viscosity within the range 11250-21000 cP and a substitution degree of methoxy groups of 19-24% by weight and of hydroxypropoxy groups of 7-12% by weight.

HPMC (50 cP) has a viscosity within the range 37.5-70 cP and a substitution degree of methoxy groups of 28-30% by weight and of hydroxypropoxy groups of 7-12% by weight.

The viscosity values are given for 2% (w/w) aqueous solutions at 20° C.

The active substance, (R)-3-N,N-dicyclobutylamino-8-fluoro-3,4-dihydro-2H-1-benzopyran-5-carboxamide hydrogen (2R,3R)-tartrate monohydrate, HPC and the two different HPMC were screened through a 1.0 mm square screen and mixed in a Turbula mixer for 2 minutes at 46 rpm. The powder mixture was granulated with water in a planetary mixer for 1+1 minutes. The wet granulation was dried in a drying cabinet at 45° C. for 15 hours. The granulation was milled through a screen of 1.27 mm. The dry powder mass was mixed with magnesium stearate (screened through 0.5 mm) in a Turbula mixer for 2 minutes at 46 rpm. Colloidal silicon dioxide was added through a screen of 0.5 mm and the mixing was continued for further 2 minutes.

The final homogeneous powder mixture was compressed into tablets in a tableting machine equipped with a normally curved punch of Ø 8 mm.

In order to test the release of the active drug substance from the tablet an in vitro dissolution of the tablet was accomplished by using the USP paddle method with 50 rpm.

(Dissolution Test, USP 24 p. 1941)

Used conditions:

Medium: Phosphate buffer, pH=6.8, 500 ml, Temperature: 37° C.

The following results were obtained:

| Time | Amount |
|---|---|
| (h) | dissolved % |
| 0 | 0 |
| 0.5 | 14 |
| 1 | 20 |
| 2 | 30 |
| 4 | 46 |
| 8 | 66 |
| 12 | 81 |
| 18 | 94 |
| 24 | 105 |

### Example 2

The following components, expressed as mg per tablet, were used; batch size 2000 tablets:

| | |
|---|---|
| Active substance | 5.00 |
| HPC LF | 3.5 |
| HPMC (15000 cP) | 118.0 |
| HPMC (50 cP) | 50.0 |
| Paraffin special | 18.0 |
| Water | 110.2 |
| Magnesium stearate | 2.7 |
| Colloidal silicon dioxide | 0.9 |

The tablet in Example 2 was produced in the same manner as in Example 1.

The in vitro release of the active substance, (R)-3-N,N-dicyclobutylamino-8-fluoro-3,4-dihydro-2H-1-benzopyran-5-carboxamide hydrogen (2R,3R)-tartrate monohydrate, from the dosage form of Example 2 was obtained using the same method as in Example 1.

The following results were obtained:

| Time | Amount |
|---|---|
| (h) | dissolved % |
| 0 | 0 |
| 0.5 | 15 |
| 1 | 23 |
| 2 | 34 |
| 4 | 51 |
| 8 | 73 |
| 12 | 86 |
| 18 | 99 |

### Example 3

The following components, expressed as mg per tablet, were used; batch size 5000 tablets:

| | |
|---|---|
| Active substance: | 5 |
| HPC LF | 3.5 |
| HPMC (15000 cP) | 200 |
| HPMC (50 cP) | 86 |
| Water | 110.2 |
| Magnesium stearate | 4.5 |
| Collodial silicon dioxide | 1.5 |

The active substance, (R)-3-N,N-dicyclobutylamino-8-fluoro-3,4-dihydro-2H-1-benzopyran-5-carboxamide hydrogen (2R,3R)-tartrate monohydrate, lactose powder, HPMC (15000 cP) and HPC-LF were screened through a 1.0 mm screen and mixed inTurbula mixer for 10 minutes. The powder mixture was granulated with water in an intensive mixer for 2 minutes. The wet granulation was dried in a drying cabinet at 45° C. for about 15 hours. The granulation was milled through a screen of 1.0 mm in an oscillating mill. The milled granulation was mixed with colloidal silicon dioxide and magnesium stearate (both screened through 0.5 mm) in a Turbula mixer for 2 minutes. The final homogeneous powder mixture was compressed into tablets in an eccentric single punch tableting machine equipped with a normally curved punch of Ø 9 mm.

The in vitro release of the active substance, (R)-3-N,N-dicyclobutylamino-8-fluoro-3,4-dihydro-2H-1-benzopyran-5-carboxamide hydrogen (2R,3R)-tartrate monohydrate, from the dosage form of Example 3 was obtained using the same method as in Example 1.

The following results were obtained:

| Time (h) | Amount dissolved % |
|---|---|
| 0 | 0 |
| 1 | 13 |
| 2 | 22 |
| 4 | 35 |
| 8 | 53 |
| 12 | 67 |
| 16 | 76 |
| 20 | 84 |
| 24 | 90 |

Conclusion:

From the Examples 1 to 3 it is evident that with the tablets according to the present invention a continuous extended release of the active substance is obtained up to 24 hours.

### Example 4

The following components, expressed as mg per tablet, were used; batch size 5000 tablets:

| | |
|---|---|
| Active substance | 5 |
| Lactose powder | 90 |
| HPMC (15000 cP) | 72 |
| HPC LF | 3.5 |
| Water | 110 |
| Colloidal silicon dioxide | 0.9 |
| Magnesium stearate | 2.7 |

The tablet in Example 4 was produced in the same manner as in Example 3, with the exception that the punch diameter was 8 mm.

In order to test the release of the active substance from the tablet an in vitro dissolution of the tablet was accomplished by using the USP Paddle method at 50 rpm. (Dissolution test, USP 24 p.1941)

Medium: Phosphate buffer, pH=6.8, 500 ml, Temperature: 37° C.,

The following results were obtained:

| Time (h) | Amount dissolved (%) |
|---|---|
| 0 | 0 |
| 1 | 28 |
| 2 | 46 |
| 4 | 69 |
| 6 | 85 |
| 8 | 93 |
| 10 | 98 |
| 12 | 99 |

The result obtained from Example 4 show that addition of lactose as a release modifying agent enhances the in vitro release from 100% release after 20 to 24 hours to 100% release after 10 to 12 hours.

### Example 5

The following components, expressed as mg per tablet, were used; batch size 2850 tablets:

| | |
|---|---|
| Active substance | 5 |
| HPMC (15000 cP) | 250 |
| PVP | 25 |
| Water | 100 |
| Microcrystalline cellulose | 55 |
| Colloidal silicon dioxide | 1.6 |
| Sodium stearyl fumarate | 3.3 |
| COATING | |
| HPMC (6 cP) | 4.2 |
| PEG (6000) | 1.0 |
| Titanium dioxide | 1.2 |
| Water | 115 |

HPMC (15000 cP) has a viscosity within the range of 11250-21000 cP and a substitution degree of methoxy groups of 19-24% by weight and hydroxypropoxy groups of 7-12% by weight. The viscosity values are given for 2% (w/w) aqueous solutions at 20° C.

The active substance, (R)-3-N,N-dicyclobutylamino-8-fluoro-3,4-dihydro-2H-1-benzopyran-5-carboxamide hydrogen (2R,3R)-tartrate monohydrate, HPMC (15000 cP) and polyvinylpyrrolidone were sieved through a 0.5 mm screen (or 1.0 mm screen for HPMC and PVP) and were mixed in a Turbula mixer for 10 minutes at a speed of 33 rpm. The powder mixture was granulated with water in an intensive mixer during water addition and additionally mixed for 2 minutes. The wet granulation was dried in a drying cabinet at 45° C. for 10 hours. The granulation was milled through a screen of 1.25 mm in an oscillating mill at 147 rpm. The granulation was compacted into loose compacts in a tablet machine equipped with punches of Ø 11 mm. The compacts were milled through a screen of 4 mm and then through a screen of 1.25 mm. The milled granulation was mixed with microcrystalline cellulose and colloidal silicon dioxide (screened through 0.5 mm) in a

Turbula mixer for 6 minutes at a speed of 33 rpm. Sodium stearyl fumarate was added through a 0.5 mm screen and the mixing was continued for further 2 minutes.

The final homogeneous powder mixture was compressed into tablets in a tablet machine equipped with normally curved punches of Ø 10 mm.

The tablets were spray-coated in a tablet coating machine using an aqueous coating suspension of HPMC (6 cP) and PEG6000 and high speed homogenised suspended titanium dioxide.

In order to test the release of the active substance, (R)-3-N,N-dicyclobutylamino-8-fluoro-3,4-dihydro-2H-1-benzopyran-5-carboxamide hydrogen (2R,3R)-tartrate monohydrate, from the tablet an in vitro dissolution of the tablet was accomplished by using the USP Paddle method at 50 rpm. (Dissolution test, USP 24 p.1941)

Medium: Phosphate buffer, pH=6.8, 500 ml, Temperature: 37° C.

The following results were obtained:

| Time (h) | Amount dissolved (%) |
|---|---|
| 0 | 0 |
| 1 | 16 |
| 2 | 25 |
| 5 | 44 |
| 10 | 63 |
| 15 | 76 |
| 20 | 85 |
| 24 | 90 |

Bioavailability

A single dose, two-way crossover bioavailability study was performed in 18 healthy volunteers. Two different extended release formulations of (R)-3-N,N-dicyclobutylamino-8-fluoro-3,4-dihydro-2H-1-benzopyran-5-carboxamide hydrogen (2R,3R)-tartrate monohydrate were tested. Two parallel groups of fasting volunteers received (R)-3-N,N-dicyclobutylamino-8-fluoro-3,4-dihydro-2H-1-benzopyran-5-carboxamide hydrogen (2R,3R)-tartrate monohydrate in aqueous solution the first day and as an extended release tablet according to Example 3 or 4 the following day. A third parallel group received single doses of (R)-3-N,N-dicyclobutylamino-8-fluoro-3,4-dihydro-2H-1-benzopyran-5-carboxamide hydrogen (2R,3R)-tartrate monohydrate the first day as an aqueous solution and the following day as an extended release tablet according to Example 3, both together with food (high fat standardised breakfast). The subjects received either 5 mg (R)-3-N,N-dicyclobutylamino-8-fluoro-3,4-dihydro-2H-1-benzopyran-5-carboxamide hydrogen (2R,3R)-tartrate monohydrate in a tablet or 2.5 mg (R)-3-N,N-dicyclobutylamino-8-fluoro-3,4-dihydro-2H-1-benzopyran-5-carboxamide hydrogen (2R,3R)-tartrate monohydrate in an aqueous solution after overnight fasting. Plasma samples were withdrawn prior to and up to 24 hours after drug administration. Determination of (R)-3-N,N-dicyclobutylamino-8-fluoro-3,4-dihydro-2H-1-benzopyran-5-carboxamide hydrogen (2R,3R)-tartrate monohydrate in the plasma was performed using HPLC. The area under the plasma concentration of (R)-3-N,N-dicyclobutylamino-8-fluoro-3,4-dihydro-2H-1-benzopyran-5-carboxamide hydrogen (2R,3R)-tartrate monohydrate versus time curve (AUC₀₋₂₄), the maximum plasma concentration (Cₘₐₓ), the time to reach the maximum blood plasma concentration (tₘₐₓ), the mean residence time (MRT) and the relative bioavailability (Fᵣₑₗ) were calculated. The results are presented in Tables A and B below.

TABLE A
Pharmacokinetic data obtained after administration of the
extended release oral dosage form of Example 3.

| | Aqueous | | | Aqueous | | |
|---|---|---|---|---|---|---|
| | solution | ER_{24h,fasting} | ER_{24h,fasting}/ | solution | ER_{24h,food} | ER_{24h,food}/ |
| | (2.5 mg) | (5 mg) | Aq. sol. | (2.5 mg) | (5 mg) | Aq. sol. |
| Parameter | Fasting | Fasting | Fasting | Food | Food | Food |
| Cₘₐₓ (nmol/l) | 119.0 | 45.8 | | 113.0 | 84.5 | |
| tₘₐₓ (h) | 0.67 | 4.0 | 6.0 | 1.1 | 6.0 | 5.5 |
| AUC₀₋₂₄ | 303 | 512 | | 518 | 962 | |
| (nmol * h/l) | | | | | | |
| MRT (h) | 2.5 | 11.2* | 4.5 | 3.6 | 12.3* | 3.4 |
| Fᵣₑₗ | | 0.81 | | | 0.93 | |

*estimated MRT

TABLE B
Pharmacokinetic data obtained after administration of the
extended release oral dosage form of Example 4.

| | Aqueous | | |
|---|---|---|---|
| | solution | ER_{12 h} | ER_{12 h}/ |
| | (2.5 mg) | (5 mg) | Aq. Sol. |
| Parameter | Fasting | Fasting | Fasting |
| Cₘₐₓ (nmol/l). | 149.0 | 82.7 | |
| tₘₐₓ (h) | 0.33 | 2.0 | 6.1 |
| AUC₀₋₂₄ | 328 | 629 | |
| (nmol*h/l) | | | |
| MRT (h) | 2.2 | 8.2* | 3.7 |
| Fᵣₑₗ | | 0.95 | |

*estimated MRT

The results show that the extended release oral dosage form according to the present invention provides for a defined plasma concentration of the active substance over a predetermined period of time. Further, it is shown that the extended release oral dosage forms according to the present invention provides a blood plasma profile of the active substance with a prolonged time for the maximum peak concentration (tₘₐₓ). The time to reach the maximum blood plasma concentration (tₘₐₓ) is at least five times as long as the tₘₐₓ obtained when the active substance is administered orally in an aqueous solution. The rapid initial rise in blood plasma concentration is prevented by administration of the active substance in the extended release dosage form of the present invention.

The results also show that the MRT increases at least three times when the active substance is administered orally in the extended release oral dosage form of the present invention compared to the MRT obtained after orally administration of the active substance in an aqueous solution.

Further, there is no dose dumping when the active substance is administered in the extended release oral dosage form of the present invention.

1. An extended release oral dosage form comprising the active substance (R)-3-N,N-dicyclobutylamino-8-fluoro-3,4-dihydro-2H-1-benzopyran-5-carboxamide in the form of a free base, hydrate, and/or solvate, or a pharmaceutically acceptable salt thereof, in mixture with at least one gel-forming polymer and optionally one or more excipients.

2. The extended release oral dosage form according to claim 1, wherein the salt of (R)-3-N,N-dicyclobutylamino-8-fluoro-3,4-dihydro-2H-1-benzopyran-5-carboxamide is (R)-3-N,N-dicyclobutylamino-8-fluoro-3,4-dihydro-2H-1-benzopyran-5-carboxamide hydrogen (2R,3R)-tartrate.

3. The extended release oral dosage form according to claim 1, wherein the salt of (R)-3-N,N-dicyclobutylamino-8-fluoro-3,4-dihydro-2H-1-benzopyran-5-carboxamide is (R)-3-N,N-dicyclobutylamino-8-fluoro-3,4-dihydro-2H-1-benzopyran-5-carboxamide hydrogen (2R,3R)-tartrate monohydrate.

4. The extended release oral dosage from according to claim 1, wherein the gel-forming polymer is selected from the group consisting of hydroxymethyl cellulose, hydroxypropyl cellulose, hydroxyethyl cellulose, hydroxypropyl methylcellulose (HPMC), methyl cellulose, ethyl cellulose, polyvinylpyrrolidone, polyethylene glycols, polyethylene oxides and poloxamers.

5. The extended release oral dosage form according to claim 4, wherein the gel-forming polymer is HPMC having a viscosity in the range of from 3000 to 21000 cP, a substitution degree of methoxy groups in the range of from 19 to 30% by weight, and a substitution degree of hydroxypropoxy groups in the range of from 4 to 12% by weight.

6. The extended release oral dosage form according to claim 5, wherein the gel-forming HPMC is in admixture with a low viscosity HPMC, the low viscosity HPMC having a viscosity in the range of from 3.75 to 140 cP, a substitution degree of methoxy groups in the range of from 19 to 30% by weight, and a substitution degree of hydroxypropoxy groups in the range of from 4 to 12% by weight.

7. The extended release oral dosage form according to claim 1, wherein the binder is selected from the group consisting of hydroxypropyl cellulose, glycerylbehenate, microcrystalline cellulose, polyvinylpyrrolidone, and mixtures thereof.

8. The extended release oral dosage form according to claim 1, wherein the lubricant is selected from the group consisting of magnesium stearate powder, sodium stearyl fumarate, stearic acid, polyethylene glycol, and talc.

9. The extended release oral dosage form according to claim 1, wherein the release modifying agent is selected from the group consisting of lactose, mannitol, sorbitol, calcium phosphate, aluminium silicate, paraffin, carboxypolymethylene, carboxyvinyl polymer, acrylic acid polymer, ethyl cellulose, and polyethylene glycol.

10. (Canceled)

11. The extended release oral dosage form according to claim 1, wherein the ratio of the active substance to gel-forming polymer is in the range of from 1:10 to 1:60.

12. The extended release oral dosage form according to claim 1, wherein the dosage form comprises a binder, and the ratio of the active substance to binder is in the range of from 1:0.5 to 1:5.

13. The extended release oral dosage form according to claim 1, wherein the amount of the active substance in the dosage form is less than 10% w/w.

14. The extended release oral dosage form according to claim 1, wherein the dosage form further comprises a coating layer.

15. The extended release oral dosage form according to claim 14, wherein the coating layer optionally comprises one or more substances selected from the group consisting of plasticizers, colour agents, pigments, taste-masking agents, and anti-adhesion agents.

16. The extended release oral dosage form according to claim 1, wherein the dosage form is uncoated.

17-33. (Canceled)

34. The extended release oral dosage form according to claim 1, wherein the dosage form has a mean dissolution profile in vitro, in phosphate buffer at a pH of 6.8, using the USP Paddle method at 50 rpm, such that about 30 to 75% of the active substance is released after 4 hours, about 60 to 100% is released after 12 hours and about 80 to 100% is released after 24 hours.

35. The extended release oral dosage form according to claim 34, wherein the dosage form has a mean dissolution profile in vitro, in phosphate buffer at a pH of 6.8, using the USP Paddle method at 50 rpm, such that about 30 to 55% of the active substance is released after 4 hours, about 60 to 90% is released after 12 hours and about 80 to 100% is released after 24 hours.

36. The extended release oral dosage form according to claim 34, wherein the dosage form has a mean dissolution profile in vitro, in phosphate buffer at a pH of 6.8, using the USP Paddle method at 50 rpm, such that about 50 to 75% of the active substance is released after 4 hours, about 70 to 95% is released after 8 hours and about 90 to 100% is released after 12 hours.

37. The extended release oral dosage form according to claim 1, wherein the dosage form upon administration provides a release of the active substance over a period of up to 24 hours, wherein the time to reach the maximum blood plasma concentration (tₘₐₓ) of the active substance is at least five times as long as the tₘₐₓ obtained when the active substance is administered orally in an aqueous solution.

38. The extended release oral dosage form according to claim 1, wherein the time to reach the maximum blood plasma concentration (tₘₐₓ) of the active substance is at least 3 hours.

39. The extended release oral dosage form according to claim 1, wherein the dosage form upon administration provides a mean residence time (MRT) of the active substance that is at least three times as long as the MRT obtained when the active substance is administered orally in an aqueous solution.

40. The extended release oral dosage form according to claim 1, wherein the mean residence time (MRT) of the active substance is between 8 and 15 hours.

41. The extended release oral dosage form according to claim 1, wherein the one or more excipients are selected from the group consisting of binders, lubricants, release modifying agents, and flow condition agents.

42. The extended release oral dosage form according to claim 41, wherein the flow condition agent is colloid silicon dioxide.

43. The extended release oral dosage form according to claim 5, wherein the gel-forming HPMC has a viscosity between 7500 and 21000 cP.

44. The extended release oral dosage form according to claim 5, wherein the gel-forming HPMC has a viscosity between 11250 and 21000 cP.

45. The extended release oral dosage form according to claim 5, wherein the gel-forming HPMC has a substitution degree of methoxy groups in the range of from 19 to 28%.

46. The extended release oral dosage form according to claim 5, wherein the gel-forming HPMC has a substitution degree of methoxy groups in the range of from 19 to 24%.

47. The extended release oral dosage form according to claim 5, wherein the gel-forming HPMC has a substitution degree of hydroxypropoxy groups in the range of from 7 to 12% by weight.

48. The extended release oral dosage form according to claim 6, wherein the low viscosity HPMC has a viscosity in the range of from 11.3 to 140 cP.

49. The extended release oral dosage form according to claim 6, wherein the low viscosity HPMC has a viscosity in the range of from 37.5 to 70 cP.

50. The extended release oral dosage form according to claim 6, wherein the low viscosity HPMC has a substitution degree of methoxy groups in the range of from 19 to 28% by weight.

51. The extended release oral dosage form according to claim 6, wherein the low viscosity HPMC has a substitution degree of methoxy groups in the range of from 19 to 24% by weight.

52. The extended release oral dosage form according to claim 6, wherein the low viscosity HPMC has a substitution degree of hydroxypropoxy groups in the range of from 7 to 12% by weight.

53. The extended release oral dosage form according to claim 11, wherein the ratio of the active substance to gel-forming polymer is in the range of from 1:30 to 1:60.

54. The extended release oral dosage form according to claim 12, wherein the ratio of the active substance to binder is in the range of from 1:0.7 to 1:5.

55. The extended release oral dosage form according to claim 14, wherein the coating layer comprises a polymer selected from the group consisting of hydroxypropyl cellulose, polyethylene glycol, low viscosity HPMC, and mixtures thereof.

56. The extended release oral dosage form according to claim 40, wherein the MRT is between 8 and 13 hours.

57. A process for the manufacture of an extended release dosage form according to any one of claims 1-9, 11-16, or 34-56, the process selected from the group consisting of:
method A, comprising the steps of:
Ai) mixing the active substance with at least one gel-forming polymer and optionally one or more pharmaceutically excipients,
Aii) forming the obtained dry powder mixture into a solid dosage form, and
Aiii) optionally coating the obtained dosage form;
method B, comprising the steps of:
Bi) mixing the active substance with at least one gel-forming polymer and optionally one or more pharmaceutically acceptable excipients,
Bii) granulating the mixture,
Biii) optionally drying the obtained granulate,
Biv) mixing the granulate with one or more pharmaceutically acceptable excipients,
Bv) forming the obtained dry powder mixture into a solid dosage form, and
Bvi) optionally coating the obtained dosage form; and
method C, comprising the steps of:
Ci) mixing the active substance with at least one gel-forming polymer and optionally one or more pharmaceutically acceptable excipients,
Cii) granulating the mixture,
Ciii) optionally drying the obtained granulate powder mass,
Civ) compressing the granulate powder mass into loose compacts,
Cv) milling the compacts and mixing the milled compacts with one or more pharmaceutically excipients,
Cvi) forming the obtained dry powder mixture into a solid dosage form, and
Cvii) optionally coating the obtained dosage form.

58. The process according to claim 57, wherein the granulation in step Bii and Cii is performed in water.

59. A method for the prophylaxis and/or treatment of a medical condition selected from the group consisting of disorders and related medical disturbances in the central nervous system, urinary incontinence, vasospasm, and growth control of tumours, the method comprising administering an effective amount of the extended release oral dosage form according to any one of claims 1-9, 11-16, or 34-56 to a patient in need thereof.

60. The method according to claim 59, wherein the medical condition is a 5-hydroxy-tryptamine-mediated disorder or disturbance.

61. The method according to claim 59, wherein the medical condition is depression, anxiety, or a memory disorder.

62. The method according to claim 59, wherein the medical condition is a disturbance of the cardiovascular system or a disturbance of the gastrointestinal system.

63. The method according to claim 59, wherein the medical condition is over-active urine bladder.

64. The method according to claim 61, wherein the medical condition is Alzheimer's Disease.
